(19) United States
(12) Patent Application Publication
(10) Pub. No.: US 2009/0239900 A1
(43) Pub. Date: Sep. 24, 2009

(54) QUININE AND QUINIDINE SALTS, METHODS FOR MAKING THEM, AND PHARMACEUTICAL FORMULATIONS COMPRISING THEM

(75) Inventors: Jean Paul REMON, Melle (BE); Chris Vervaet, Izegem (BE)

Correspondence Address: CLARK & ELBING LLP, 101 FEDERAL STREET, BOSTON, MA, 02110, US

(73) Assignee: Universiteit Gent, Gent (BE)

(21) Appl. No.: 12/053,357

(22) Filed: Mar. 21, 2008

(51) Int. Cl. A61K 31/439; A61P 33/06; A61P 21/02

(52) U.S. Cl. 514/305

(57) ABSTRACT

The present invention provides a combination of:
a N-containing drug molecule salt, wherein said drug molecule is selected from the group consisting of quinine and quinidine, and
an aromatic mono- or dicarboxylic acid salt,
   e.g. quinine pamoate, which is useful for a pharmaceutical formulation such as a pediatric suspension, especially in the treatment of malaria.

The present invention relates to novel quinine and quinidine salts with lower water-solubility characteristics than known quinine and quinidine salts. The present invention also relates to methods for producing these novel poorly water-soluble quinine and quinidine salts. The present invention also relates to pharmaceutical compositions including effective amounts of such novel poorly water-soluble quinine and quinidine salts, exhibiting unexpectedly improved taste-masking characteristics. The present invention also relates to methods of treatment of malaria, leg cramps and restless legs syndrome by the administration of an effective dose of such novel poorly water-soluble quinine and quinidine salts, e.g. in the form of an aqueous suspension formulation.

### BACKGROUND OF THE INVENTION

Quinine is the last chance of treatment of multi-drug resistant or severe malaria. In children, an oral dose of 10 mg quinine hydrochloride or sulphate per kilogram body weight every eight hours for seven days is recommended. However, no pediatric formulations containing quinine hydrochloride or sulphate are commercially available. This may be due to the fact that quinine is a very bitter drug, which reduces compliance in children. The same concerns more or less apply to quinidine, a quinine isomer with the same utility.

In the United States of America, quinine sulfate is indicated only for treatment of uncomplicated Plasmodium falciparum malaria in adults at a dosage of 648 mg every eight hours for seven days, but is not approved for treating severe or complicated P. falciparum malaria, for preventing malaria or for the treatment or prevention of nocturnal leg cramps. This is apparently due to serious adverse events associated with the use of available quinine pharmaceutical formulations based on presently available quinine salts (cardia arrythmia, thrombocytopenia, cinchonism, gastrointestinal troubles, ocular problems including permanent bilateral visual loss), potentially serious interactions with other drugs, and the fact that presently available quinine pharmaceutical formulations exhibit a narrow margin between an effective dose and a toxic dose.

Quinine overdose can be associated with serious complications, including visual impairment, hypoglycemia, cardiac arrhythmias, and death. Visual impairment can range from blurred vision and defective color perception, to visual field constriction and permanent blindness. Cinchonism occurs in virtually all patients with quinine overdose. Symptoms range from headache, nausea, vomiting, abdominal pain, diarrhea, tinnitus, vertigo, hearing impairment, sweating, flushing, and blurred vision, to deafness, blindness, serious cardiac arrhythmias, hypotension, and circulatory collapse. Central nervous system toxicity (drowsiness, disturbances of consciousness, ataxia, convulsions, respiratory depression and coma) has also been reported with quinine overdose, as well as pulmonary edema and adult respiratory distress syndrome.

There is therefore a need in the art for novel specific pharmaceutically acceptable forms of quinine which either could be effective against various forms of malaria at lower doses than the presently available quinine sulfate formulations or would show a reduced frequency of adverse events and complications with respect to the presently available quinine sulfate formulations. Provided these goals would be met, such novel pharmaceutically acceptable forms of quinine would consequently open a safer way to other therapeutic indications, including leg cramps and related diseases such as, but not limited to, restless legs syndrome and akathisia.

Taste masking of bitter drugs like quinine and quinidine for oral administration to human beings, especially children, usually requires the design of an aqueous drug suspension of a poorly water-soluble drug. In the case of quinine and quinidine, such a desirable pharmaceutically acceptable, in particular pediatric, formulation is made especially difficult, due to the rather high water-solubility of the active drugs or their commonly available salts. For instance the water-solubility limit of quinine and quinidine is about 0.5 g/L in cold water. The water-solubility of most common quinine salts is even higher, and the bitterness of their crystals or powders is also much higher. Thus, the challenge of formulating a low soluble drug is combined here with the challenge of taste masking bitterness of the drug.

There is therefore also a need in the art for specific pharmaceutically acceptable forms of quinine and quinidine exhibiting a lower water-solubility and/or a lower bitterness than the presently existing forms, thus making such forms more accessible for taste masking in drug formulations, especially in pharmaceutical aqueous suspensions, and more especially in pediatric pharmaceutical formulations.

Completion of a 7-day oral quinine treatment may be limited by drug intolerance, due to one or more of the above stated adverse events and complications of the presently available quinine sulfate formulations. Consequently shorter courses (3 days) of quinine therapy in combination with one or more other anti-malarial agents such as, but not limited to, tetracycline, doxycycline, or clindamycin, have been used. Consequently, there is also a need in the art for specific forms of quinine and quinidine which, in addition to the above-listed advantages, can be combined with one or more other anti-malarial agents under specific circumstances.

There is also a need in the art for specific forms of quinine and quinidine which, in addition of exhibiting the above-listed advantageous characteristics, can be produced by using practical manufacturing methods which are easy to upscale to any desired production amount, and inexpensively.

There is also a need in the art for specific forms of quinine and quinidine which, in addition of exhibiting the above-listed advantageous characteristics and easiness of industrial production at any scale, can be easily formulated into pharmaceutical compositions, either in solid forms or as liquid suspensions, providing at least the same therapeutic efficacy as the presently existing forms of quinine and quinidine, and showing a better compliance or comfort in patients, especially in children, due to a reduced bitterness and/or a substantial taste-masked effect.

All these needs constitute a series of problems addressed by the present invention. As a consequence, this invention should provide a safe, patient-compliant and more effective treatment of multi-drug resistant or severe forms of malaria, especially in children, as well as a safe, patient-compliant and effective treatment of other diseases such as, but not limited to, leg cramps, restless legs syndrome and akathisia.

Although the present invention is mainly focused on improving existing forms of quinine and quinidine, it may be conveniently applicable to other N-containing drug molecules exhibiting the same or a similar combination of bitterness and high water-solubility characteristics.

### SUMMARY OF THE INVENTION

The present invention is based on the unexpected finding that the above needs in the art can be efficiently and inexpensively met by combining quinine or quinidine with an aromatic mono- or di-carboxylic acid moiety. This combination may take the form of combining a quinine or quinidine salt, preferably a quinine or quinidine inorganic salt, more preferably a quinine or quinidine salt derived from a strong inorganic acid, with an aromatic mono- or dicarboxylic acid salt, preferably an aromatic mono- or dicarboxylic acid alkali or alkaline-earth metal salt. This combination may easily be produced by contacting, via dispersion in a liquid medium, a quinine or quinidine salt (including any preferred form thereof, as mentioned hereinabove) together with an aromatic mono- or dicarboxylic acid salt (including any preferred form thereof, as mentioned hereinabove) under conditions, especially respective amounts of the contacted reactants and period of contacting time, sufficient for forming a precipitate of said combination. In this way, the combination product can easily be separated from the liquid medium and, if desired, purified until any required level of purity.

### BRIEF DESCRIPTION OF THE DRAWINGS

FIG. 1 shows the respective dissolution profiles of quinine from a quinine pamoate suspension in hydrochloric acid acid 0.1 N (♦), a pH 5.8 phosphate buffer (□) and demineralized water ().

FIG. 2 shows the bioavailbility of quinine liquid formulations in dogs, respectively for a quinine pamoate suspension according to an embodiment of this invention, before (▪) and after stomach acidification (∘), and for a quinine hydrochloride solution (▴) of the prior art.

### DETAILED DESCRIPTION OF THE INVENTION

The present invention is based on the improved taste masking effect in mammals, especially in humans, and more especially in children, obtained by combining quinine or quinidine with an aromatic mono- or dicarboxylic acid moiety such as, but not limited to, a pamoic acid moiety, into a “quinine combination product” or a “quinidine combination product”. Other aromatic mono- or dicarboxylic acid moieties that impart such improved taste masking effect to quinine or quinidine may be selected from the group consisting of biphenylene-2-carboxylic acid, 1H-indene-3-carboxylic acid, anthracene-9-carboxylic acid, anthracene-1-carboxylic acid, anthracene-2-carboxylic acid, 3-hydroxyanthracene-2-carboxylic acid, 1-hydroxy-2-naphthoic acid, 6-hydroxy-2-naphthoic acid, 3-hydroxy-2-naphthoic acid, 2-hydroxy-1-naphthoic acid, 5-hydroxy-1-naphthoic acid, 6-hydroxy-1-naphthoic acid, and isomers thereof. Thus the skilled person is able to appropriately select, depending upon parameters such as, but not limited to, the desired bioavailibility profile, the cost of the aromatic mono- or dicarboxylic acid reactant, the easiness (yield and contacting time) of the combination, the nature and the number of any optional pharmaceutically acceptable excipients with which the “quinine combination product” or “quinidine combination product” may be formulated into a medicament, an optimal aromatic mono- or dicarboxylic acid moiety.

In one embodiment of the present invention, the combination may take the form of combining:
a quinine or quinidine salt, preferably a quinine or quinidine inorganic salt, more preferably a quinine or quinidine salt derived from a strong inorganic acid, most preferably a halide such as a hydrochloride or hydrobromide, or a sulphate of quinine or quinidine, or a hydrate thereof, with
an aromatic mono- or dicarboxylic acid salt, preferably an aromatic mono- or dicarboxylic acid alkali or alkaline-earth metal salt, more preferably a pamoic acid alkali metal salt, most preferably pamoic acid disodium.

More specifically, the quinine or quinidine salt included in the combination product according to the present invention may be any commercially available quinine or quinidine salt, or hydrate thereof, such as but not limited to quinine sulphate, quinine sulphate dihydrate, quinine monohydrochloride, quinine monohydrochloride dihydrate, quinine bisulfate, quinine bisulfate heptahydrate, quinine hydrobromide, quinine dihydrochloride, quinine dihydrobromide, quinidine hydrochloride, quinidine hydrochloride monohydrate and quinidine sulfate dihydrate (cinquine).

In another embodiment of the present invention, the combination may take more specific forms such as, but not limited to, quinine pamoate, quinine biphenylene-2-carboxylate, quinine 1H-indene-3-carboxylate, quinine anthracene-9-carboxylate, quinine anthracene-1-carboxylate, quinine anthracene-2-carboxylate, quinine 3-hydroxyanthracene-2-carboxylate, quinine 1-hydroxy-2-naphthoate, quinine 6-hydroxy-2-naphthoate, quinine 3-hydroxy-2-naphthoate, quinine 2-hydroxy-1-naphthoate, quinine 5-hydroxy-1-naphthoate, quinine 6-hydroxy-1-naphthoate, quinidine pamoate, quinidine biphenylene-2-carboxylate, quinidine 1H-indene-3-carboxylate, quinidine anthracene-9-carboxylate, quinidine anthracene-1-carboxylate, quinidine anthracene-2-carboxylate, quinidine 3-hydroxyanthracene-2-carboxylate, quinidine 1-hydroxy-2-naphthoate, quinidine 6-hydroxy-2-naphthoate, quinidine 3-hydroxy-2-naphthoate, quinidine 2-hydroxy-1-naphthoate, quinidine 5-hydroxy-1-naphthoate, quinidine 6-hydroxy-1-naphthoate, and the like, in particular isomers thereof.

According to another embodiment of the present invention, the “quinine combination product” or “quinidine combination product” may readily be formed via anion exchange in a liquid medium, i.e. the anion from an inorganic acid (e.g. chloride, bromide or sulphate) is exchanged with an aromatic mono- or dicarboxylate anion. This may be conveniently and expediently achieved by selecting a liquid medium in which the desired “quinine combination product” or “quinidine combination product” is insoluble and precipitates, therefore allowing for easy recovery, and later purification if desired. The combination products may thus easily be produced by contacting, in the form of a dispersion in a liquid medium, a quinine or quinidine salt (e.g. a halide or a sulphate), or a hydrate thereof, together with an aromatic mono- or dicarboxylic acid salt (e.g. an alkali or alkaline-earth metal salt, more preferably a sodium or potassium salt) under conditions such as, but not limited to, respective amounts of the contacted reactants, type of liquid dispersing medium, concentrations of the contacted reactants in the liquid dispersing medium, temperature and period of contacting time, sufficient for forming a precipitate of said combination product. All such reaction conditions can easily be optimised in view of the result, e.g. reaction yield and product characteristics (such as, but not limited to, average particle size and/or particle size distribution), to be achieved.

Preferred respective amounts of the contacted reactants depend upon the type of anions involved, e.g. whether a mono- or dicarboxylic acid salt is selected as the aromatic acid reactant, but usually include molar equivalent amounts or, preferably, a slight molar excess of the mono- or dicarboxylic acid salt (e.g. in the case of a dicarboxylic acid, a quinine/dicarboxylic acid molar ratio from about 2/1 to about 2/0.4, optimally a 2/1.2 molar ratio). Preferred concentrations of the contacted reactants in the liquid dispersing medium typically range from about 1 g/L to about 6 g/L when water is used as the liquid dispersing medium. Alternative liquid dispersing media include predominantly aqueous media.

Preferred contacting temperatures typically range from about 15° C. to about 45° C. Preferred contacting times typically range from about 30 seconds to about 60 minutes. The skilled person readily understands that the contacting time can be terminated as soon as complete precipitation of the resulting “quinine combination product” or “quinidine combination product” has been observed.

Although the resulting average particle size and/or particle size distribution characteristics of the “quinine combination product” or “quinidine combination product” of this invention may depend upon the conditions used for its preparation, such as above listed, it has been found that the combination product of this invention may easily be obtained in the form of particles with an average particle size from about 3 μm to about 20 μm.

The desired “quinine combination product” or “quinidine combination product”, after in situ precipitation in the liquid dispersing medium, may be post-treated in any suitable way, depending upon the intended final use. If the intended use of the “quinine combination product” (e.g. quinine pamoate) or “quinidine combination product” according to this invention is a liquid pharmaceutical composition such as a pediatric suspension e.g. for administration to children diagnosed with multi-drug resistant or severe malaria, it may be unnecessary to isolate the precipitate formed. Formulating the liquid pharmaceutical composition may merely involve stirring the precipitate suspension, together with addition of one or more viscosity enhancing agents and optionally other pharmaceutically acceptable excipients until the desired target viscosity is achieved for the suspension.

If the intended use of the “quinine combination product” according to this invention (e.g. quinine pamoate) or “quinidine combination product” is an oral dosage solid form, the precipitate can be used as such or can be washed with a suitable washing medium such as water, and then dried under drying conditions (including drying temperature and drying time) conventional in the art for such N-containing drug molecules, and eventually converted or incorporated into an oral dosage solid form such as a tablet, a capsule, granules or pellets, together with addition of one or more pharmaceutically acceptable excipients suitable for the selected oral dosage solid form, until the desired solid form characteristics (e.g. tablet friability and/or compressibility, or others) and/or the desired drug release characteristics are achieved.

Hard gelatin capsules are known as a conventional pharmaceutical solid dosage form. Their sizes have been standard since the start of industrial manufacture of drug compositions, ranging from 5 (corresponding to a volume of 0.13 ml) up to 000 (corresponding to a volume of 1.36 ml). Thus, when a large amount of active ingredient (e.g. a quinine or quinidine combination product of this invention) is required for each dosage unit, depending on the bulk density of the formulation, it may be necessary to use large size capsules.

Coated active ingredient (e.g. a quinine or quinidine combination product of this invention) tablets are also of interest. This may include producing tablets comprising microcapsules, due to the advantages resulting from the microencapsulated substance being protected from external influences and vice-versa (e.g. increased stability, reduced chances of irritations or undesirable reactions with other components in a mixture, ability to mask unpleasant tastes and smells), although compaction of coated beads or pellets for making tablets may be difficult. As is well known in the pharmaceutical industry, beads or pellets are quite distinguishable from granules. Beads can be defined as small, free-flowing spherical or sphere-like particles manufactured by pelletization, i.e. the agglomeration of fine powders or granules of drug substances and excipients using appropriate processing equipment. As opposed to the process of granulation, producing beads by pelletization results in a larger average size and a narrower size-range distribution and may also be applicable to the formulation of a “quinine combination product” (e.g. quinine pamoate) or a “quinine combination product” according to this invention into an oral dosage solid form.

The solid dosage forms of this invention may be prepared using any method known in the art for manufacturing similar pharmaceutical compositions and may comprise one or more pharmaceutically acceptable additives such as, but not limited to, sweeteners, flavouring agents, colouring agents, preservatives and the like. Other suitable carrier materials and excipients are detailed below and may include inter alia calcium carbonate, sodium carbonate, lactose, calcium phosphate or sodium phosphate; granulating and disintegrating agents, binding agents and the like. The pharmaceutical compositions of this invention may be included in a hard gelatin capsule in admixture with one or more inert solid diluents or carrier materials.

The term “pharmaceutically acceptable carrier or excipient” as used herein in relation to pharmaceutical compositions refers to any material or substance with which the active principle, i.e. the quinine combination product (e.g. quinine pamoate), may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders.

Suitable pharmaceutical carriers for use in the said pharmaceutical formulations are well known to those skilled in the art. There is no particular restriction to their selection within the present invention although special attention may be paid to the selection of suitable carrier combinations that can assist in properly formulating the quinine combination product (e.g. quinine pamoate) in view of the expected time release profile. Suitable pharmaceutical carriers include additives such as wetting agents, dispersing agents, stickers, adhesives, emulsifying or surface-active agents, thickening agents, viscosity enhancing agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals, in particular humans.

The pharmaceutical compositions of the present invention may be prepared in any known manner, for instance by homogeneously mixing, dissolving, spray-drying, coating and/or grinding the active ingredient, in a one-step or a multi-steps procedure, together with the selected carrier material and, where appropriate, the other additives such as surface-active agents. They may also be prepared by encapsulation, for instance in view to obtain microspheres (usually having a diameter of about 1 to 10 μm), namely for the manufacture of microcapsules for controlled or sustained release of the active ingredient.

Suitable surface-active agents to be used in the pharmaceutical compositions of the present invention may be non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates and alkaline or alkaline-earth metal salts thereof, or optionally substituted ammonium salts thereof.

Suitable non-ionic surfactants include, but are not limited to, polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing from 3 to 10 glycol ether groups and from 8 to 20 carbon atoms in the (aliphatic) hydrocarbon moiety and from 6 to 18 carbon atoms in the alkyl moiety of the alkylphenol. Further suitable non-ionic surfactants include, but are not limited to, water-soluble adducts of polyethylene oxide with polypropylene glycol, nonylphenolpolyethoxyethanol, fatty acid esters of polyethylene sorbitan, and the like.

Suitable cationic surfactants include, but are not limited to, quaternary ammonium salts (preferably halides) having four hydrocarbon radicals optionally substituted with halogeno, phenyl, substituted phenyl or hydroxyl; and quaternary ammonium salts containing at least one C₈-C₂₂ alkyl radical (e.g. cetyl, lauryl, palmityl, myristyl, oleyl and the like) and at least one optionally halogenated C₁₋₄ alkyl, benzyl and/or hydroxy-C₁₋₄ alkyl radical.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants (Chemical Publishing Co., N.Y., 1981).

Structure-forming, thickening or gel-forming agents may be included into the pharmaceutical compositions of the present invention. Suitable such agents include in particular, but are not limited to, highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to about 20 carbon atoms; cetostearyl alcohol and modified castor oil products (e.g. the product commercially available under the trade name Antisettle).

Gelling (or viscosity enhancing) agents which may be included into the pharmaceutical compositions of the present invention include, but are not limited to, cellulose derivatives such as carboxymethylcellulose, hydroxypropylmethylcellulose and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the pharmaceutical compositions of the present invention include additives such as magnesium oxide; azo dyes; organic and inorganic pigments such as titanium dioxide; UV-absorbers; stabilisers; odour masking agents; antioxidants such as, for example, ascorbyl palmitate, sodium bisulfite, sodium metabisulfite, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavouring agents such as natural vanillin; buffers such as citric acid and acetic acid; extenders or bulking agents such as silicates, diatomaceous earth, magnesium oxide or aluminium oxide; densification agents such as magnesium salts; and mixtures thereof.

Additional ingredients may be included in order to control the duration of action of the active ingredient in the pharmaceutical compositions of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxy-methylcellulose, protamine sulfate, and mixtures thereof. The rate of drug release and duration of action may also be controlled by methods such as incorporating the active ingredient into particles, e.g. microcapsules, of a polymeric substance such as hydrogels, polylactic acid, hydroxymethyl-cellulose, polymethyl methacrylate and others. Such methods also include colloid drug delivery systems like liposomes, microspheres, microemulsions, nanoparticles, nanocapsules and so on. Depending on the route of administration, the pharmaceutical compositions of the present invention may also require protective coatings.

The combination products and salts of the present invention, and pharmaceutical compositions including them, are useful in various therapeutic areas where their taste masking effect and improved patient-compliance make them an attractive alternative to existing quinine sulfate formulations. These therapeutic areas include various forms of malaria, including uncomplicated malaria, and other diseases such as, but not limited to, leg cramps, restless legs syndrome and akathisia.

The combination products and salts of the present invention, and pharmaceutical compositions including them, may also be useful for malaria therapy in adults and children in combination with an effective dose of one or more other known anti-malarial agents such as, but not limited to, tetracycline, doxycycline, or clindamycin.

The following examples are provided solely for the purpose of illustrating the principles and advantages of the present invention but should in no way be interpreted as limiting the scope thereof, which is defined only by the claims.

### EXAMPLE 1

### Preparation of Quinine Pamoate and Pharmaceutical Suspensions Including Quinine Pamoate

2 g of quinine monohydrochloride (hereinafter referred as QHCl) were dissolved in 50 ml water. Separately, 1.44 g pamoic acid disodium (hereinafter referred as PA) were dissolved in 50 ml water. Both solutions were then combined, resulting in a QHCl/PA molar ratio of 2/1, and the mixture was stirred at room temperature for at least 30 seconds. A yellow precipitate was formed and identified as quinine pamoate.

After 10 minutes stirring the precipitate, a viscosity enhancing agent (0.2% weight/volume xanthan gum, or 1% weight/volume sodium carboxymethylcellulose commercially available under the trade name Avicel® RC581) was added, and stirring was continued for 20 more minutes, thus resulting in quinine pamoate suspensions with a viscosity suitable for oral administration.

### EXAMPLE 2

### Physical Characterization and Biological Testing of Quinine Pamoate Suspensions

The quinine pamoate suspensions from example 1 were tested for taste masking efficiency, dissolution profile, and bioavailability in dogs.

Taste masking efficiency was tested by the analysis of free quinine in the supernatant by high performance liquid chromatography. The amount of free quinine was found to be 0.08 mg/L, and it was also found that by modifying the recipe of example 1 by changing the QHCl/PA molar ratio to 2/1.2, this amount can still be decreased to 0.01 mg/L.

Taste masking efficiency was tested by five volunteers (3 male, 2 female, age below 30 years) who found the suspension of example 1 to be tasteless.

The particle size and sedimentation characteristics of quinine pamoate particles in the suspensions of example 1 were determined in triplicate by laser diffraction by means of a Mastersizer-S apparatus from Malvern (United Kingdom) equipped with 300 RF lens and a mixing system set at 1,500 rpm. The particle size distributions found for each suspension, before and after long-term storage under specific temperature conditions, are indicated in the following table (wherein RT means room temperature):

TABLE
suspension stability testing

| Viscosity | Storage | | Size (μm) distribution | | |
|---|---|---|---|---|---|
| enhancer | conditions | | D(v, 0.1) | D(v, 0.5) | D(v, 0.9) |
| Xanthan gum | Before | | 0.5 | 9.3 | 19.8 |
| | 3 months | RT | 0.4 | 7.3 | 15.8 |
| | | 40° C. | 0.4 | 8.4 | 18.0 |
| | 6 months | RT | 1.2 | 6.2 | 17.3 |
| | | 40° C. | 3.7 | 10.9 | 22.3 |
| Avicel ® | Before | | 5.7 | 21.5 | 70.4 |
| RC 581 | 3 months | RT | 6.2 | 24.1 | 78.4 |
| | | 40° C. | 5.3 | 26.4 | 86.2 |
| | 6 months | RT | 7.2 | 26.0 | 85.8 |
| | | 40° C. | 3.3 | 28.8 | 86.6 |

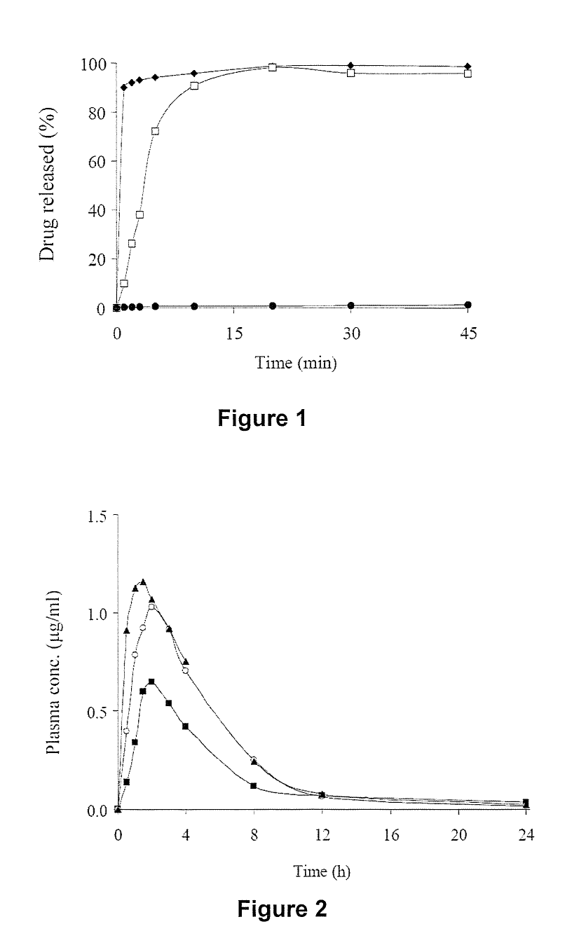

The dissolution profile of the suspension of example 1 was determined in accordance with the dissolution testing method XXVII of United States Pharmacopeia, both in chlorhydric acid at different concentrations, and in water). Results are shown in FIG. 1.

The bioavailability in dogs of the suspension of example 1, as such (shown as U in FIG. 2) and after stomach acidification using 6 mg/kg pentagastrine (shown as ▪ in FIG. 2), was evaluated, and compared to that of a quinine hydrochloride aqueous solution (shown as ▴ in FIG. 2), in six fasting dogs following pentagastrine pretreatment to lower the stomach pH. 6 μg/kg pentagastrine was injected intramuscularly one hour before drug intake. The studies were organised in a randomised cross-over design. Each dog was randomly assigned to receive a single dose of quinine pamoate suspension or a freshly prepared quinine hydrochloride solution equivalent to 8.2 mg/kg. A washout period of 1 week separated both drug intakes. Venous blood samples were taken before, and respectively 0.5, 1, 1.5, 2, 3, 4, 8, 12 and 24 hours after drug intake. Plasma samples were analysed for quinine using a validated HPLC method. Results are shown in FIG. 2, indicating that higher plasma concentrations and high bioavailability of the quinine pamoate suspension of example 1 were observed after gastric acidification.

All publications and patent applications mentioned in this specification are herein incorporated by reference to the same extent as if each independent publication or patent application was specifically and individually indicated to be incorporated by reference.

Other embodiments are within the scope of the claims.

What is claimed is:

1. The combination of:
a N-containing drug molecule salt, wherein said drug molecule is selected from the group consisting of quinine and quinidine, and
an aromatic mono- or dicarboxylic acid salt.

2. The combination of claim 1, wherein said aromatic mono- or dicarboxylic acid is pamoic acid.

3. The combination of claim 1, wherein said aromatic mono- or dicarboxylic salt is an alkali or alkaline-earth metal salt.

4. The combination of claim 1, wherein:
said aromatic mono- or dicarboxylic salt is a sodium salt, and
said aromatic mono- or dicarboxylic acid is pamoic acid.

5. The combination of claim 1, comprising a salt of said N-containing drug molecule, the anion of said salt being an aromatic mono- or dicarboxylate.

6. A pharmaceutical composition comprising one or more pharmaceutically acceptable excipients and a combination of:
a N-containing drug molecule salt, wherein said drug molecule is selected from the group consisting of quinine and quinidine, and
an aromatic mono- or dicarboxylic acid salt.

7. A pharmaceutical composition according to claim 6, wherein said one or more pharmaceutically acceptable excipients comprise one or more viscosity enhancing agents.

8. A pharmaceutical composition according to claim 6, wherein said one or more pharmaceutically acceptable excipients comprise a viscosity enhancing effective amount of xanthan gum and/or sodium carboxymethylcellulose.

9. A pharmaceutical composition according to claim 6, in the form of a suspension in one or more pharmaceutically acceptable liquid carriers.

10. A pharmaceutical composition according to claim 6, in the form suitable for pediatric oral administration.

11. A pharmaceutical composition according to claim 6, in the form of a tablet, capsule, pellet or powder.

12. A method for producing a combination according to claim 1, comprising contacting, in dispersion in a liquid medium:
a N-containing drug molecule salt, wherein said drug molecule is selected from the group consisting of quinine and quinidine, and
an aromatic mono- or dicarboxylic acid salt,
under conditions sufficient for forming a precipitate of said combination.

13. A method of treatment of a disease selected from the group consisting of malaria, leg cramps, restless legs syndrome and akathisia, comprising the administration of an effective amount of a combination according to claim 1 to a patient in need thereof.

14. A method of treatment of malaria in children, comprising the administration of an effective amount of a combination according to claim 1, said combination being in the form of a suspension in one or more pharmaceutically acceptable liquid carriers.

15. A method of treatment of malaria in children, comprising the administration of an effective amount of a combination according to claim 1, said combination being in the form of a suspension in one or more pharmaceutically acceptable liquid carriers and further comprising one or more viscosity enhancing agents.

16. A method of treatment of malaria in children, comprising the administration of an effective amount of a combination according to claim 1, said combination being in the form of a tablet, a hard gelatine capsule or a powder.

17. A method of treatment of malaria in children, comprising the administration of an effective amount of a combination according to claim 1, wherein said quinine pamoate therapeutic dosage is administered together with an effective dose of one or more other anti-malarial agents.
